(19) United States
(12) Patent Application Publication
(10) Pub. No.: US 2009/0328240 A1
(43) Pub. Date: Dec. 31, 2009

(54) Genetically modified mice as predictors of immune response

(76) Inventors: George L. Sing, New York, NY (US); Linda O. Palladino, Stormville, NY (US)

Correspondence Address: LINDA O. PALLADINO, 45 HONEYSUCKLE COURT, STORMVILLE, NY, 12582, US

(21) Appl. No.: 12/456,722

(22) Filed: Jun. 22, 2009

Related U.S. Application Data
(60) Provisional application No. 61/132,942, filed on Jun. 24, 2008.

(51) Int. Cl. A61K 49/00; A01K 67/027; A61P 43/00

(52) U.S. Cl. 800/3; 800/18

(57) ABSTRACT

The invention is directed to novel genetically modified organisms and uses thereof. In particular, the invention is directed to novel genetically modified mice and uses of such mice to assess the immunogenic potential of human therapeutic antigens and to predict immune responses.

### CROSS-REFERENCE TO RELATED APPLICATIONS

This application claims priority under 35 USC §119(e) of U.S. Provisional Application No. 61/132,942, filed Jun. 24, 2008, the entirety of which is incorporated herein by reference.

### FIELD OF THE INVENTION

The field of the invention is directed to novel genetically modified organisms and uses thereof. In particular, the field of the invention is directed to novel genetically modified mice and uses of such mice to assess the immunogenic potential of human therapeutic antigens and to predict immune responses.

### DESCRIPTION OF RELATED ART

VaxDesign Corporation, located at 12612 Challenger Parkway, Suite 365, Orlando, Fla. 32826 (vaxdesign.com), is a biotechnology company that develops high-throughput in vitro assays of the human immune system that are designed to be functionally equivalent to the human immune system, and are intended to be used to predict human responses to pharmaceuticals and vaccines.

U.S. Pat. No. 6,596,541, issued Jul. 22, 2003, describes the replacement, in whole or in part, in a non-human eukaryotic cell, the endogenous immunoglobulin variable region gene locus with an homologous or orthologous human immunoglobulin variable gene locus. This replacement utilizes the methodology described in U.S. Pat. No. 6,586,251, issued Jul. 1, 2003, which, briefly, describes a method for genetically modifying an endogenous gene or chromosomal locus of interest in isolated eukaryotic cells, comprising: a) obtaining a large cloned genomic fragment greater than 20 kb containing a DNA sequence of interest; b) using bacterial homologous recombination to genetically modify the large cloned genomic fragment of (a) to create a large targeting vector for use in eukaryotic cells (LTVEC), such LTVEC having homology arms which total greater than 20 kb; c) introducing the LTVEC of (b) into the isolated eukaryotic cells to modify by homologous recombination the endogenous gene or chromosomal locus in the cells; and d) using a quantitative assay to detect modification of allele (MOA) in the eukaryotic cells of (c) to identify those eukaryotic cells in which the endogenous gene or chromosomal locus has been genetically modified.

### BACKGROUND OF THE INVENTION

Many drugs that appear to be efficacious in animal models ultimately fail in human clinical trials. Failure may be due to toxicity, lack of efficacy in humans, immune response to the therapeutic agent, or a combination of these reasons. Much effort has been directed to finding in vitro and preclinical in vivo assays and models to more accurately assess the likelihood of success of a therapeutic agent before millions of dollars are invested in human clinical trials. One particular area of interest is in designing in vitro and in vivo models to help predict the immunogenicity of a therapeutic agent. Interestingly, there are instances where immunogenicity is desirable (i.e. vaccine development) as well as instances when it is undesirable (i.e. immune response resulting in neutralization of a therapeutic agents, for example, neutralization of a protein).

The major histocompatability complex (MHC) is a large genomic region or gene family found in most vertebrates. It is the most gene-dense region of the mammalian genome and plays an important role in the immune system, autoimmunity, and reproductive success. The proteins encoded by the MHC are expressed on the surface of cells and display both self antigens and non-self antigens to T cells that have the capacity to kill or coordinate the killing of pathogens, infected or malfunctioning cells.

In humans, the 3.6 Mb MHC region is located on chromosome 6 and contains 140 genes. About half of these genes have known immunological functions. The MHC region is divided into three subgroups called MHC class I, MHC class II, and MHC class III. The MHC class I region encodes heterodimeric peptide-binding proteins, as well as antigen-processing molecules such as TAP and Tapasin. The MHC class II region encodes heterodimeric peptide-binding proteins and proteins that modulate antigen loading onto MHC class II proteins in the lysosomal compartment such as MHC class II DM, MHC class II DQ, MHC class II DR, and MHC class II DP. The MHC class III region encodes for other immune components, such as complement components (e.g., C2, C4, factor B) and some that encode cytokines (e.g., TNF-α) and also hsp.

The best-known genes in the MHC region are the subset that encodes cell-surface antigen-presenting proteins. In humans, these genes are referred to as human leukocyte antigen (HLA) genes. The most intensely studied HLA genes are the nine so-called classical MHC genes: HLA-A, HLA-B, HLA-C, HLA-DPA1, HLA-DPB1, HLA-DQA1, HLA-DQB1, HLA-DRA, and HLA-DRB1. The A, B, and C genes belong to MHC class I, whereas the six D genes belong to MHC class II.

One of the most striking features of the MHC, particularly in humans, is its allelic diversity, especially among the nine classical genes. In humans, HLA-A, HLA-B, and HLA-DRB1 have roughly 250, 500, and 300 known alleles, respectively.

It is well established in the literature that an individual's HLA class II alleles impact that individual's response to various antigenic stimuli. For example, Johnson, A. H., et al., (2004, Infect Immun 72(5):2762-2771) report that human leukocyte antigen class II alleles influence levels of antibodies to the Plasmodium falciparum asexual-stage apical membrane antigen 1 but not to merozite surface antigen 2 and merozite surface protein 1, and Poland, G. A., et al., (2001, Vaccine 20(3-4):430-438) report on the identification of an association between HLA class II alleles and low antibody levels after measles immunization.

There are in vitro systems that aim to address the issue of immunogenicity and an individual's response to particular antigens. For example, VaxDesign Corporation (12612 Challenger Parkway, Suite 365, Orlando, Fla. 32826), has technology which is attempting to mimic the human immune system with in vitro assays designed to predict human responses to pharmaceuticals and vaccines. However, in vitro systems, while useful, are generally thought to fall short of the prediction that could be possible using an appropriate in vivo model.

Therefore, it is an object of the subject invention to provide an in vivo model system that is capable of more accurately predicting human response to antigen by integrating the diversity of the human MHC class II region into the mouse genome.

### BRIEF SUMMARY OF THE INVENTION

Applicants describe, for the first time, a novel in vivo murine model system, termed “MuResponse”, which utilizes a panel of genetically modified mice to predict the immune response human subjects may have to an antigen. The MuResponse system is designed such that each MuResponse mouse in the panel has been genetically modified to contain the human HLA class II genetic locus that corresponds to a particular human subpopulation having a same or similar locus. For example, it has been estimated that approximately 80% of the Caucasian population falls into ˜11 representative loci combinations. The MuResponse^{C} panel of mice has been engineered to encompass the loci covering all of these combinations present in the Caucasian population. Similarly, the MuResponse^{Af}, MuResponse^{As}, MuResponse^{H} encompass the most common loci in African Americans, Asians and Hispanics, respectively. Thus, by testing an antigen in the appropriate MuResponse panel of mice, it becomes possible to predict which HLA class II genotypes are more or less likely to mount an immune response to the antigen. In the case of vaccines, an increased immune response would indicate that a particular HLA class II genotype subpopulation is more likely to benefit from the vaccination then an HLA class II genotype subpopulation that exhibits a reduced or absent immune response. Conversely, if the MuResponse panel of mice exposed to an antigen, for example a protein-based therapeutic, revealed that mice with a certain HLA class II genotype mount an immune response, but others did not, one could target drug treatment to the corresponding human subpopulation that did not mount the response, thus avoiding the cost and safety issues associated with treating patients with a drug from which they will not derive a benefit and which could cause them harm. This would also serve to help design clinical trials such that subjects whose HLA class II genotype predicts an immune response would be excluded from the trial, thus saving millions of clinical trial costs and providing results that more accurately represent efficacy.

Accordingly, a first aspect of the invention is a genetically modified mouse, wherein such genetic modification is replacement of the mouse H-2 class II locus with a human HLA class II locus.

A second aspect of the invention is the genetically modified mouse of aspect one which is useful for determining the immune response a human population may have to an antigen.

A third aspect of the invention is the genetically modified mouse of aspect one, wherein the human HLA class II locus is selected from Caucasian, African American, Asian or Hispanic human populations.

A fourth aspect of the invention is the genetically modified mouse of aspect three wherein the human HLA class II locus is selected from a subpopulation of a Caucasian, African American, Asian or Hispanic population.

A fifth aspect of the invention is the method for determining the immune response a Caucasian, African American, Asian or Hispanic subject may have to an antigen comprising administering the antigen to a mouse of aspect four and observing whether an immune response occurs in the mouse.

A sixth aspect of the invention is a genetically modified mouse, wherein such genetic modification is accomplished by injecting the nucleus from a human AMP cell into an enucleated mouse ES cell or blastocyst cell and allowing the resulting cell or blastocyst to develop into the genetically modified mouse.

A seventh aspect of the invention is the genetically modified mouse of aspect six wherein the HLA class II haplotype of the human AMP cell is determined prior to injection into the ES cell or blastocyst cell.

An eighth aspect of the invention is the genetically modified mouse of aspect seven wherein the human HLA class II haplotype is selected from a subpopulation of a Caucasian, African American, Asian or Hispanic population.

A ninth aspect of the invention is the method for determining the immune response a Caucasian, African American, Asian or Hispanic subject may have to an antigen comprising administering the antigen to a mouse of aspect eight and observing whether an immune response occurs in the mouse.

A tenth aspect of the invention is the method of determining the likelihood a human subject will have an immune response to an antigen comprising a) determining the HLA class II genotype of the human subject; b) administering the antigen to a genetically modified mouse having the same/similar HLA class II haplotype as the human subject; and c) observing whether an immune response occurs in the mouse when it is exposed to the antigen.

Other features and advantages of the invention will be apparent from the accompanying description, examples and the claims. The contents of all references, pending patent applications and published patents, cited throughout this application are hereby expressly incorporated by reference. In case of conflict, the present specification, including definitions, will control.

### DEFINITIONS

As used herein, the term “targeting vector” is a DNA construct that contains sequences “homologous” to endogenous chromosomal nucleic acid sequences flanking a desired genetic modification(s). The flanking homology sequences, referred to as “homology arms”, direct the targeting vector to a specific chromosomal location within the genome by virtue of the homology that exists between the homology arms and the corresponding endogenous sequence and introduce the desired genetic modification by a process referred to as “homologous recombination”.

As used herein, the term “homologous” means two or more nucleic acid sequences that are either identical or similar enough that they are able to hybridize to each other or undergo intermolecular exchange.

As used herein, the term “gene targeting” is the modification of an endogenous chromosomal locus by the insertion into, deletion of, or replacement of the endogenous sequence via homologous recombination using a targeting vector.

As used herein, the term “gene knockout” is a genetic modification resulting from the disruption of the genetic information encoded in a chromosomal locus.

As used herein, the term “gene knockin” is a genetic modification resulting from the replacement of the genetic information encoded in a chromosomal locus with a different DNA sequence.

As used herein, the term “knockout organism” is an organism in which a significant proportion of the organism's cells harbor a gene knockout.

As used herein, the term “knockin organism” is an organism in which a significant proportion of the organism's cells harbor a gene knockin.

As used herein, the term “marker” or a “selectable marker” is a selection marker that allows for the isolation of rare transfected cells expressing the marker from the majority of treated cells in the population. Such marker's gene's include, but are not limited to, neomycin phosphotransferase and hygromycin B phosphotransferase, or fluorescing proteins such as GFP.

As used herein, the term “ES cell” is an embryonic stem cell. This cell is usually derived from the inner cell mass of a blastocyst-stage embryo.

As used herein, the term “ES cell clone” is a subpopulation of cells derived from a single cell of the ES cell population following introduction of DNA and subsequent selection.

As used herein, the term “flanking DNA” is a segment of DNA that is collinear with and adjacent to a particular point of reference.

As used herein, the term “non-human organism” is an organism that is not normally accepted by the public as being human.

As used herein, the term “Orthologous” sequence refers to a sequence from one species that is the functional equivalent of that sequence in another species.

As used herein, the term “genetically modified” means a DNA molecule which has been manipulated such that is contains nucleotide sequences that are not normally found in that DNA molecule. For example, manipulating mouse DNA molecules such that they contain human nucleotide sequences.

A “transgenic mammal” as used herein refers to an animal containing one or more cells bearing genetic information, received, directly or indirectly, by deliberate genetic manipulation at the subcellular level, such as by microinjection or transfection with recombinant DNA, or infection with recombinant virus.

The term “germ cell-line transgenic animal” refers to a transgenic animal in which the genetic information was introduced into a germ line cell, thereby conferring the ability to transfer the information to offspring. If such offspring in fact possess the transgene, they too are transgenic mammals.

As used herein, the term “MuResponse” means a panel of genetically modified mice in which each mouse's DNA has been manipulated such that it contains a particular human MHC class II region. A MuResponse panel may be constructed for any desired population. For example, the MuResponse^{C} panel of mice has been engineered to encompass the loci covering all of the human MHC class II allele combinations present in the Caucasian population. Similarly, the MuResponse^{Af}, MuResponse^{As}, MuResponse^{H} encompass the most common human MHC class II allele combinations in African Americas, Asians and Hispanics, respectively.

As used herein, the term “human HLA class II locus”, “human HLA class II region” or “human HLA class II genotype” means the segment of human DNA encoding the genes for HLA-DPA1, HLA-DPB1, HLA-DQA1, HLA-DQB1, HLA-DRA, and HLA-DRB1.

As used herein, the term “mouse H-2 class II locus”, “mouse H-2 class II region” or “mouse H-2 class II genotype” means the segment of mouse DNA encoding the genes for H-2-A, and H-2-E. The mouse H-2 locus is on mouse chromosome 17.

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells that are either in an undifferentiated, partially differentiated, or fully differentiated form, or a combination thereof, into a human or other animal.

As used herein, the terms “a” or “an” means one or more; at least one.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984,“Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, the preferred methods and materials are now described.

It must be noted that as used herein and in the appended claims, the singular forms “a,” “and” and “the” include plural references unless the context clearly dictates otherwise.

Generation of Genetically Modified (MuResponse) Mice

A. Human HLA class II loci representative of major human subpopulations—Table 1 sets for the 11 most common MHC class II gene haplotypes found in Caucasians.

TABLE 1
11 Most Common DR-DQ Haplotypes in Caucasian Americans (C = Caucasian)

| MuResponse^{C} | DR | DR-DQ | DR | DQ | | |
|---|---|---|---|---|---|---|
| Panelist # | Serotype | Haplotype | B1 | A1 | B1 | Frequency (%) |
| C1 | DR1 | DR1-DQ5 | 0101 | 0101 | 0501 | 9.1 |
| C2 | DR3 | DR3-DQ2 | 0301 | 0501 | 0201 | 13.1 |
| C3 | DR4 | DR4-DQ7 | 0401 | 0300 | 0301 | 5.4 |
| C4 | DR4 | DR4-DQ7 | 0401 | 0300 | 0302 | 5.0 |
| C5 | DR4 | DR4-DQ8 | 0404 | 0300 | 0392 | 3.9 |
| C6 | DR7 | DR7-DQ2 | 0701 | 0201 | 0202 | 11.1 |
| C7 | DR7 | DR7-DQ9 | 0701 | 0201 | 0303 | 3.7 |
| C8 | DR10 | DR10-DQ5 | 1101 | 0505 | 0301 | 5.6 |
| C9 | DR13 | DR13-DQ6 | 1301 | 0103 | 0603 | 5.6 |
| C10 | DR13 | DR13-DQ6 | 1302 | 0102 | 0604 | 3.4 |
| C11 | DR15 | DR15-DQ6 | 1501 | 0102 | 0602 | 14.2 |

B. Generation of targeting vectors—Gene targeting by means of homologous recombination between homologous exogenous DNA and endogenous chromosomal sequences has proven to be an extremely valuable way to create deletions, insertions, design mutations, correct gene mutations, introduce transgenes, or make other genetic modifications in mice. Current methods involve using standard targeting vectors, with regions of homology to endogenous DNA typically totaling less than 10-20 kb, to introduce the desired genetic modification into mouse embryonic stem (ES) cells, followed by the injection of the altered ES cells into mouse embryos to transmit these engineered genetic modifications into the mouse germline (Smithies et al., Nature, 317:230-234, 1985; Thomas et al., Cell, 51:503-512, 1987; Koller et al., Proc Natl Acad Sci USA, 86:8927-8931, 1989; Kuhn et al., Science, 254:707-710, 1991; Thomas et al., Nature, 346:847-850, 1990; Schwartzberg et al., Science, 246:799-803, 1989; Doetschman et al., Nature, 330:576-578, 1987; Thomson et al., Cell, 5:313-321, 1989; DeChiara et al., Nature, 345:78-80, 1990; U.S. Pat. No. 5,789,215, issued Aug. 4, 1998 in the name of GenPharm International). In addition, particularly well-suited methodologies are described in U.S. Pat. No. 6,586,251 and U.S. Pat. No. 6,596,541.

In addition to ES cells, pluripotent stem cells derived from the late epiblast of mouse embryos, called Epiblast stem cells, (see Brons, I.G.M., et al., Nature 2007, 448(12):191-197; Tesar, P.J., et al, Nature 2007, 448(12):196-199) are also suitable for use in creating the MuResponse mice of the invention, as are the AEC^{R}, ADC^{R} and AMP^{R} cells described in U.S. Provisional Application No. 61/205,235, filed Jan. 20, 2009, or any cell which has been reprogrammed to pluripotency, such cells generally referred to as iPCs or induced pluripotent cells. Any of the above methodologies and cells are useful for creating the MuResponse mice of the invention. All of the aforementioned references are incorporated herein in their entirety.

C. Identification of correctly targeted non-human cells used in the methods—Skilled artisans are familiar with techniques used to identify correctly targeted non-human cells. For example, detecting the rare cells in which the standard targeting vectors have correctly targeted and modified the desired endogenous gene(s) or chromosomal locus(loci) requires sequence information outside of the homologous targeting sequences contained within the targeting vector. Assays for successful targeting involve standard Southern blotting or long PCR (Cheng, et al., Nature, 369:684-5, 1994; Foord and Rose, PCR Methods Appl, 3:S149-61, 1994; Ponce and Micol, Nucleic Acids Res, 20:623, 1992; U.S. Pat. No. 5,436,149 issued to Takara Shuzo Co., Ltd.) from sequences outside the targeting vector and spanning an entire homology arm; thus, because of size considerations that limit these methods, the size of the homology arms are restricted to less than 10-20 kb in total (Joyner, The Practical Approach Series, 293, 1999). In addition, particularly well-suited methodologies for identifying correctly targeted non-human cells are described in U.S. Pat. No. 6,586,251 and U.S. Pat. No. 6,596,541 (Such approaches can include but are not limited to: (a) quantitative PCR using TaqMan™. (Lie and Petropoulos, Curr Opin Biotechnol, 9:43-8, 1998); (b) quantitative MOA assay using molecular beacons (Tan, et al., Chemistry, 6:1107-11, 2000) (c) fluorescence in situ hybridization FISH (Laan, et al., Hum Genet, 96:275-80, 1995) or comparative genomic hybridization (CGH) (Forozan, et al., Trends Genet, 13:405-9, 1997; Thompson and Gray, J Cell Biochem Suppl, 139-43, 1993; Houldsworth and Chaganti, Am J Pathol, 145:1253-60, 1994); (d) isothermic DNA amplification (Lizardi, et al., Nat Genet, 19:225-32, 1998; Mitra and Church, Nucleic Acids Res, 27:e34, 1999); and (e) quantitative hybridization to an immobilized probe(s) (Southern, J. Mol. Biol. 98: 503, 1975; Kafatos F C; Jones C W; Efstratiadis A, Nucleic Acids Res 7(6):1541-52, 1979). All of the aforementioned references are incorporated herein in their entirety.

D. Microinjection of nuclei isolated from amnion-derived multipotent progenitor (AMP) cells into enucleated mouse ES and/or blastocyst cells—In one embodiment of the invention, using standard technologies, nuclei obtained from AMP cells (see U.S. Publication No. 2006-0222634 and U.S. Publication No. 2007-0231297 for a description of AMP cells, each reference being incorporated herein in its entirety) are injected into enucleated mouse ES cells and/or blastocyst cells to generate MuResponse mice. Prior to removal of the nuclei from the AMP cells, the cells may be tested to determine their HLA class II haplotype so that representative haplotype from all of the desired human subpopulations are identified. Once the donor AMP cell haplotypes are established, the nuclei are removed from the AMP cells and injected into the EC cell or blastocysts cells. The panel of mice generated therefrom will then encompass all major human HLA class II haplotypes for the desired subpopulation of the panel being constructed (i.e. MuResponse^{C}, MuResponse^{Af}, MuResponse^{As}, MuResponse^{H}, etc.). Nuclei from any of the other cells described above are suitable for microinjection as well.

E. Implantation of targeted non-human cells or ES cells containing AMP cell or other cell nuclei into mice—The MuResponse mice can be generated by several different techniques including standard blastocyst injection technology or aggregation techniques (Robertson, Practical Approach Series, 254, 1987; Wood, et al., Nature, 365:87-9, 1993; Joyner, The Practical Approach Series, 293, 1999), tetraploid blastocyst injection (Wang, et al., Mech Dev, 62:137-45, 1997), or nuclear transfer and cloning (Wakayama, et al., Proc Natl Acad Sci U S A, 96:14984-9, 1999). ES cells derived from other organisms such as rabbits (Wang, et al., Mech Dev, 62:137-45, 1997; Schoonjans, et al., Mol Reprod Dev, 45:439-43, 1996) or chickens (Pain, et al., Development, 122:2339-48, 1996) or other species should also be amenable to genetic modification(s) using the methods of the invention. 2. Modified protoplasts can be used to generate genetically modified plants (for example see U.S. Pat. No. 5,350,689 “Zea mays plants and transgenic Zea mays plants regenerated from protoplasts or protoplast-derived cells”, and U.S. Pat. No. 5,508,189 “Regeneration of plants from cultured guard cell protoplasts” and references therein). 3. Nuclear transfer from modified eukaryotic cells to oocytes to generate cloned organisms with modified allele (Wakayama, et al., Proc Natl Acad Sci U S A, 96:14984-9, 1999; Baguisi, et al., Nat Biotechnol, 17:456-61, 1999; Wilmut, et al., Reprod Fertil Dev, 10:639-43, 1998; Wilmut, et al., Nature, 385:810-3, 1997; Wakayama, et al., Nat Genet, 24:108-9, 2000; Wakayama, et al., Nature, 394:369-74, 1998; Rideout, et al., Nat Genet, 24:109-10, 2000; Campbell, et al., Nature, 380:64-6, 1996). 4. Cell-fusion to transfer the modified allele to another cell, including transfer of engineered chromosome(s), and uses of such cell(s) to generate organisms carrying the modified allele or engineered chromosome(s) (Kuroiwa, et al., Nat Biotechnol, 18:1086-1090, 2000).

F. Uses of MuResponse Mouse Panel—The novel in vivo murine model system, termed “MuResponse”, utilizes a panel of genetically modified mice to predict the immune response human subjects may have to an antigen. Such genetic modification my be effected by the direct modification of the mouse genome as described throughout the specification, or may be effected by microinjection of isolated nuclei from AMP cells or other desired cells into enucleated cells such as enucleated mouse ES cells. The MuResponse system is designed such that each MuResponse mouse in the panel has been genetically modified to contain the human HLA class II genetic locus that corresponds to a particular human subpopulation having a same or similar locus. The MuResponse^{C} panel of mice has been engineered to encompass the loci covering all of the combinations present in the Caucasian population. Similarly, the MuResponse^{Af}, MuResponse^{As}, MuResponse^{H} encompass the most common loci in African Americans, Asians and Hispanics, respectively. Thus, by testing an antigen in the appropriate MuResponse panel of mice, it becomes possible to predict which HLA class II genotypes are more or less likely to mount an immune response to the antigen. In the case of vaccines, an increased immune response would indicate that a particular HLA class II genotype subpopulation is more likely to benefit from the vaccination than an HLA class II genotype subpopulation that exhibits a reduced or absent immune response. Conversely, if the MuResponse panel of mice exposed to a antigen, for example a protein-based therapeutic, revealed that certain HLA class II genotype mount an immune response, but others did not, one could target drug treatment to the corresponding human subpopulation that did not mount the response, thus avoiding the cost and safety issues associated with treating patients with a drug from which they will not derive a benefit and which could cause them harm. This would also serve to help design clinical trials such that subjects whose HLA class II genotype predicts an immune response would be excluded from the trial, thus saving millions of clinical trial costs and provide results that more accurately represent efficacy.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A genetically modified mouse, wherein such genetic modification is replacement of the mouse H-2 class II locus with a human HLA class II locus.

2. The genetically modified mouse of claim 1 which is useful for determining the immune response a human population may have to an antigen.

3. The genetically modified mouse of claim 1, wherein the human HLA class II locus is selected from Caucasian, African American, Asian or Hispanic human populations.

4. The genetically modified mouse of claim 3 wherein the human HLA class II locus is selected from a subpopulation of a Caucasian, African American, Asian or Hispanic population.

5. A method for determining the immune response a Caucasian, African American, Asian or Hispanic subject may have to an antigen comprising administering the antigen to a mouse of claim 4 and observing whether an immune response occurs in the mouse.

6. A genetically modified mouse, wherein such genetic modification is accomplished by injecting the nucleus from a human AMP cell into an enucleated mouse ES cell or blastocyst cell and allowing the resulting cell or blastocyst to develop into the genetically modified mouse.

7. The genetically modified mouse of claim 6, wherein the HLA class II haplotype of the human AMP cell is determined prior to injection into the ES cell or blastocyst cell.

8. The genetically modified mouse of claim 7 wherein the human HLA class II haplotype is selected from a subpopulation of a Caucasian, African American, Asian or Hispanic population.

9. A method for determining the immune response a Caucasian, African American, Asian or Hispanic subject may have to an antigen comprising administering the antigen to a mouse of claim 8 and observing whether an immune response occurs in the mouse.

10. A method of determining the likelihood a human subject will have an immune response to an antigen comprising:
a) determining the HLA class II genotype of the human subject;
b) administering the antigen to a genetically modified mouse having the same/similar HLA class II haplotype as the human subject; and
c) observing whether an immune response occurs in the mouse.
